(19) United States
(12) Patent Application Publication
(10) Pub. No.: US 2017/0333338 A1
(43) Pub. Date: Nov. 23, 2017

(54) SUSTAINED AND REVERSIBLE ORAL DRUG DELIVERY SYSTEMS

(71) Applicant: The Children's Medical Center Corporation, Boston, MA (US)

(72) Inventors: Daniel S. Kohane, Newton, MA (US); Ryan Wylie, Ontario (CA)

(21) Appl. No.: 15/520,027

(22) PCT Filed: Oct. 20, 2015
(86) PCT No.: PCT/US2015/056469

§ 371 Date: Apr. 18, 2017

Related U.S. Application Data
(60) Provisional application No. 62/066,111, filed on Oct. 20, 2014.

(51) Int. Cl. A61K 9/00; A61K 47/34; A61K 31/663; A61K 9/107; A61K 8/86; A61Q 11/00; A61K 8/55

(52) U.S. Cl. CPC A61K 9/006; A61Q 11/00; A61K 47/34; A61K 8/55; A61K 9/107; A61K 8/86; A61K 31/663

(57) ABSTRACT

A composition that achieves sustained oral drug release is provided. The composition includes liposomes, micelles, or polymeric particles, for example, having a hydrophobic core and an amphipathic corona, wherein the core contains a therapeutic, diagnostic, nutraceutical, cosmeceutical or prophylactic agent to be released, and the corona has targeting ligands immobilized to it. In a preferred embodiment the targeting ligands are bisphosphonate molecules, which enable binding to hydroxyapatite present on teeth. The incorporation of short polyethylene glycol (PEG) chains prevents chain entanglement and ionic interactions, minimizes mucus adhesion, increases particle diffusion rates in mucus, and increases the overall stability of particles in saliva. In a preferred embodiment the agent incorporated is a dental bleaching agent and the ligands selectively bind hydroxyapatite.

### CROSS-REFERENCE TO RELATED APPLICATION

This application claims priority to and benefit of U.S. Provisional Application No. 62/066,111 filed on Oct. 20, 2014, and where permissible is incorporated by reference in its entirety.

### FIELD OF THE INVENTION

The present disclosure relates generally to non-invasive drug delivery systems, and in particular, to oral drug delivery systems.

### BACKGROUND OF THE INVENTION

Drug delivery systems (DDS) have been designed to target the mucus (mucoadhesion) of the digestive system, including the mouth. Mucus is composed of large glycoproteins that form entangled networks. In mucoadhesion, the attachment of bioadhesive particles occurs through interpenetration and non-covalent interactions. For most tissues, the mucus turnover rate limits drug delivery to only a few hours (Andrews, et al., European Journal of Pharmaceutics and Biopharmaceutics, 71:505-518 (2009); Lai, et al., Advanced Drug Delivery Reviews, 61:158-171 (2009)). Mucoadhesive systems also lack site-specificity since adhesion occurs on all mucus-covered tissues. Therefore, mucoadhesives will not provide long-term or site-specific drug delivery.

There are other problems with delivery within the mouth. For example, teeth are composed of an inner dentin layer and a hard outer enamel layer. The enamel layer, composed of the hardest and most highly mineralized substance in the body, functions to protect the teeth and provide strength. The enamel layer is composed of a translucent calcium phosphate mineral, hydroxyapatite, which forms microscopic hexagonal rods. Light passes through the tooth enamel and is reflected by the dentin, creating the normal “pearly white” appearance of teeth. Exposure to certain foods, tobacco, or coffee/tea can stimulate the formation of colored pellicle over the enamel layer, which can be removed through brushing with abrasive toothpaste or chemical treatments. Unless the pellicle layer is thoroughly removed on a regular basis, long term exposure can cause foreign material to be incorporated into the enamel, eventually causing discoloration. The stained appearance results from pores between the hydroxyapatite crystals becoming filled with foreign colored material. In addition to exposure related stains, teeth may also be naturally discolored. Methods for whitening the teeth require elaborate and frustrating efforts to apply whitener repeatedly to the teeth, or to hold trays of whiteners over the teeth for prolonged periods of time.

Currently, no product or technology exists for spatially and temporally controlled long-term delivery of drugs within the mouth, especially for the whitening of teeth

It is therefore an object of the present invention to provide a controlled release system for delivery of therapeutic, prophylactic, cosmeceutical, nutraceutical and/or diagnostic agent(s) to the mouth, especially the teeth.

### SUMMARY OF THE INVENTION

A sustained oral drug release composition has been developed that is especially useful for the delivery of therapeutic, prophylactic, cosmeceutical, nutraceutical or diagnostic agents, especially those that are not stable in saliva, have low solubility, and/or are rapidly cleared. The composition consists of polymeric particles, preferably having a hydrophobic core and an amphipathic corona, wherein the core contains a therapeutic, diagnostic, nutraceutical or prophylactic agent to be released, having targeting ligands immobilized to or within the corona, which bind to tissues in the mount or the teeth. The particles are preferably small and the corona hydrophilic or neutrally charged, to enhance penetration of mucosa. In a preferred embodiment the targeting ligands are bisphosphonate molecules, which bind to hydroxyapatite present on teeth and in bone. The additional incorporation of short polyethylene glycol (PEG) chains prevents chain entanglement and ionic interactions, minimizes mucus adhesion, increases particle diffusion rates in mucus, and increases the overall stability of particles in saliva.

In a preferred embodiment the incorporated agent is a dental bleaching agent, however, it can be a therapeutic agent, cosmeceutical, prophylactic, nutraceutical or diagnostic agent. The particles can be removed by oral administration of a solution dissociating the particles from the binding sites, such as a high pH solution.

### BRIEF DESCRIPTION OF THE DRAWINGS

FIG. 1 illustrates the oral drug delivery system (DDS). Drug-loaded particles have bisphosphonate molecules attached to their surfaces, which facilitate their binding to hydroxyapatite present on the surface of teeth. This binding is reversible by administering a solution of high pH. This DDS demonstrates localized drug delivery with temporal control.

FIG. 2 is a graph showing the time periods needed for binding of PLA-PEG-Alendronate particles to hydroxyapatite. A 48 hour incubation period was assumed to result in maximal binding and was set to 100% binding. All data were normalized to 48 hours (N=3, mean±STD).

### DETAILED DESCRIPTION OF THE INVENTION

### I. Definitions

The term “amphipathic” as used herein refers to the physical property of a molecule that possesses both hydrophilic and hydrophobic parts.

The term “bisphosphonate” as used herein refers to a compound consisting of two PO₃ (phosphonate) groups covalently linked to carbon, and constitutes a class of drugs that prevent the loss of bone mass. As such, it is often used to treat osteoporosis and similar diseases.

The term “contacting” as used herein refers to the state or condition of physical touching.

The term “continuously” as used herein refers to an event that is uninterrupted, or without cessation.

The term “hydrophilic” as used herein refers to the physical property of a molecule that is attracted to, and tends to be dissolved by, water.

The term “hydrophobic” as used herein refers to the physical property of a molecule that is seemingly repelled from a mass of water.

The term “hydroxyapatite” as used herein refers to a major component and essential ingredient of bone mineral and the matrix of teeth.

The term “nanoparticle” as used herein refers to a particle that is between 1 and up to 1000 nm in size. “Microparticles” are 1000 nm (one micron) or greater in diameter. These may be solid, have a hollow core, spherical or irregular in shape. “Particles” refers to both nano and microparticles unless otherwise specified.

### II. Particles

A. Particles

Particles are preferably formed from polymers, which can be synthetic or natural, most preferably biodegradable. Other materials include lipids, crosslinked lipids, liposomes, crosslinked liposomes, micelles, polymerosomes, and inorganic particles such as mesoporous silicates, calcium carbonate particles, polyelectrolyte based particles, and possibly particles generating hydrogen peroxide.

Polymeric Particles

Representative synthetic polymers include poly(hydroxy acids) such as poly(lactic acid), poly(glycolic acid), and poly(lactic acid-co-glycolic acid), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate.

Examples of preferred non-biodegradable polymers include ethylene vinyl acetate, poly(meth)acrylic acid, polyamides, copolymers and mixtures thereof.

In a preferred embodiment, PLGA is used as the biodegradable polymer.

The particles are designed to release molecules to be encapsulated or attached over a period of days to weeks. Factors that affect the duration of release include pH of the surrounding medium (higher rate of release at pH 5 and below due to acid catalyzed hydrolysis of hydrolytically degradable polymers such as PLGA) and polymer composition. Aliphatic polyesters differ in hydrophobicity and that in turn affects the degradation rate. The degradation rate of these polymers, and often the corresponding drug release rate, can vary from days (PGA) to months (PLA) and is easily manipulated by varying the ratio of PLA to PGA. The physiologic compatibility of PLGA and its homopolymers PGA and PLA have been established for safe use in humans for over 30 years in various human clinical applications including drug delivery systems. PLGA particles can be formulated in a variety of ways that improve drug pharmacokinetics.

The corona is formed of a hydrophilic or amphiphilic polymer. Preferred hydrophilic polymers are polyalkylene oxide polymer or copolymers. The most preferred is polyethylene glycol (“PEG”). Other compounds include PEG-polyethylene oxide (“PEO”) block copolymers such as the PLURONIC®s marketed by BASF.

Liposomes

Liposomes are biodegradable, non-toxic, unilamellar or multilamellar vesicles formed from naturally occurring or synthetic phospholipids. Liposomes have an ability to entrap and retain a wide range of therapeutic agents, either in their aqueous (hydrophilic agents) or their lipid (hydrophobic) phases (Senior, Crit. Rev. Ther. Drug Carrier Sys., 3, 123-193 (1987); Lichtenberg, Methods Biochem. Anal., 33, 337-362 (1988); Gregoriadis, Subcell. Biochem., 14, 363-378 (1989); Reimer, et al., Dermatol., 195:93 (1997)). Liposomes have been used in clinical practice for treatment of metabolic disorders (Gregoridis, et al., Prog. Clin. Biol. Res., 95, 681-701 (1982), infectious diseases (Richardson, J. Clin. Pharmacol., 29, 873-884 (1983), systemic fungal infections (Grant, et al., Biochem. Biophys. Acta, 984, 11-20 (1989) and to reduce the adverse systemic effects of chemotherapeutic drugs (Owen, et al., Anticancer Drugs, 3, 101-107 (1992); Gabizon, et al., Acta Oncol., 33, 779-786 (1994)). U.S. Pat. Nos. 7,063,860 and 8,110,217, both by Chancellor, et al., disclose liposomal delivery of capsaicin or botulinum toxin, respectively, to urothelial cells for treatment of bladder dysfunction. Twelve liposomal-therapeutic agent formulations have been approved by the U.S. Federal Drug Administration and an additional twenty-two were in clinical trials (Chang, et al., Scientific Rep., 1,195 (2012)).

Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

Liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In a preferred embodiment, the liposomes contain a phosphaditylcholine (PC) head group, and preferably sphingomyelin. In another embodiment, the liposomes contain DPPC. In a further embodiment, the liposomes contain a neutral lipid, preferably 1,2-dioleoylphosphatidylcholine (DOPC).

In certain embodiments, the liposomes are generated from a single type of phospholipid. In such embodiments, preferably the phospholipid has a phosphaditylcholine head group, and, most preferably is sphingomyelin. The liposomes may include a sphingomyelin metabolite. Sphingomyelin metabolites used to formulate the liposomes include, without limitation, ceramide, sphingosine, or sphingosine 1-phosphate. The concentration of the sphingomyelin metabolites included in the lipids used to formulate the liposomes can range from about 0.1 mol % to about 10 mol %. Preferably from about 2.0 mol % to about 5.0 mol %, and more preferably can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N″-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred. The molar ratio of a first phospholipid, such as sphingomyelin, to second lipid can range from about 5:1 to about 1:1 or 3:1 to about 1:1, more preferably from about 1.5:1 to about 1:1, and most preferably, the molar ratio is about 1:1.

The liposomes typically have an aqueous core. The aqueous core can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The liposomes have either one or several aqueous compartments delineated by either one (unilamellar) or several (multilamellar) phospholipid bilayers (Sapra, et al., Curr. Drug Deliv., 2, 369-81 (2005)). Preferably, the liposomes are multilamellar. Multilamellar liposomes have more lipid bilayers for hydrophobic therapeutic agents to associate with.

Polymersomes

Polymersomes are a class of artificial vesicles, tiny hollow spheres that enclose a solution. Polymersomes are made using amphiphilic synthetic block copolymers to form the vesicle membrane, and have radii ranging from 50 nm to 5 μm or more. Most reported polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymersomes are similar to liposomes, which are vesicles formed from naturally occurring lipids. While having many of the properties of natural liposomes, polymersomes exhibit increased stability and reduced permeability. Furthermore, the use of synthetic polymers enables designers to manipulate the characteristics of the membrane and thus control permeability, release rates, stability and other properties of the polymersome.

Several different morphologies of the block copolymer used to create the polymersomes can be utilized. The most frequently used are the linear diblock or triblock copolymers. In these cases, the block copolymer has one block that is hydrophobic; the other block or blocks are hydrophilic. Other morphologies used include comb copolymers, where the backbone block is hydrophilic and the comb branches are hydrophobic, and dendronized block copolymers, where the dendrimer portion is hydrophilic. In the case of diblock, comb and dendronized copolymers the polymersome membrane has the same bilayer morphology of a liposome, with the hydrophobic blocks of the two layers facing each other in the interior of the membrane. In the case of triblock copolymers the membrane is a monolayer that mimics a bilayer, the central block filling the role of the two facing hydrophobic blocks of a bilayer.

In general they can be prepared by the methods used in the preparation of liposomes. Film rehydration, direct injection method or dissolution method.

B. Targeting Molecules to be Attached to the Surface of the Particles

A targeting molecule is a substance which will direct the particle to a site on a selected cell or tissue type, can serve as an attachment molecule, or serve to couple or attach another molecule. Targeting molecules can be proteins, peptides, nucleic acid molecules, saccharides or polysaccharides, or low molecular weight molecules that bind to a receptor or other molecule on the surface of a targeted cell. The degree of specificity can be modulated through the selection of the targeting molecule. For example, antibodies are very specific. These can be polyclonal, monoclonal, fragments, recombinant, or single chain, many of which are commercially available or readily obtained using standard techniques.

Many targeting agents are known. Any suitable coupling method known to those skilled in the art for the coupling of ligands and polymers with double bonds, including the use of UV crosslinking, may be used for attachment of bioadhesive ligands to the polymeric microspheres described herein. Any polymer that can be modified through the attachment of lectins can be used as a bioadhesive polymer for purposes of drug delivery or imaging. Representative methods for coupling ligands to polymer and ligands for selective binding to mucosal tissue are described in U.S. Pat. No. 6,235,313

Mucosal Tissue Selective Ligands

U.S. Pat. No. 6,235,313 lists lectins that can be covalently attached to microspheres to render them target specific to the mucin and mucosal cell layer could be used as bioadhesives. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos bonus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to any microsphere may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microspheres with the appropriate chemistry, such as CDI, and be expected to influence the binding of microspheres to the gut. For example, polyclonal antibodies raised against components of mucin or else intact mucin, when covalently coupled to microspheres, would provide for increased bioadhesion. Similarly, antibodies directed against specific cell surface receptors exposed on the lumenal surface of the intestinal tract would increase the residence time of beads, when coupled to microspheres using the appropriate chemistry. The ligand affinity need not be based only on electrostatic charge, but other useful physical parameters such as solubility in mucin or else specific affinity to carbohydrate groups.

Ligands for Teeth and Bone

Hydroxyapatite, the major component of teeth, is a major component of bone. It is one of the few materials that are classified as bioactive, meaning that it will support bone ingrowth and osseointegration when used in orthopaedic, dental and maxillofacial applications. Coatings of hydroxyapatite are often applied to metallic implants such as titanium/titanium alloys and stainless steels to alter the surface properties, thereby reducing their chances of rejection by the body. Hydroxyapatite may also be employed in forms such as powders, porous blocks or beads to fill bone defects or voids. These may arise when bone augmentations are required, such as in maxillofacial reconstructions or dental applications, for instance. The bone filler provides a scaffold and encourages the rapid filling of the void by naturally forming bone and also provides an alternative to bone grafts.

Bisphosphonate ligands alendronate, neridronate, and pamidronate have affinity for hydroxyapatite (the major component of enamel), so these can be used to mediate adhesive interactions between the DDS and teeth. Adhesion to teeth by binding hydroxyapatite will be less affected by mucus turnover rates than mucoadhesiveness, and is more site-selective (Andrews, et al., Europ. J. Pharm. Biopharm. 71:505-518 (2009)). Drug delivery systems that are composed of drug-loaded particles with hydroxyapatite-targeting ligands immobilized to their surfaces facilitate binding of these particles to the tooth's surface, resulting in continuous release of an active agent, such as a dental bleaching agent. These ligands bind tightly, staying bound for at least two days under physiological conditions, and release at a pH between 9 and 11, with the most rapid and complete release at about pH 11.

Other binding agents, including antibodies or antibody fragments, that bind specifically to components such as mineral-associated adhesion molecules, such as bone morphogenic protein (BMP), bone sialoprotein (BSP) and osteopontin (OPN), can also be used.

C. Agents to be Delivered

Drug delivery systems that are composed of drug-loaded particles with targeting ligands immobilized to their surfaces facilitate binding of these particles to the mouth or tooth surface, resulting in continuous release of an active agent, such as a dental bleaching agent. The binding is reversible, and once unbound from the tooth surface, the particle will be rapidly eliminated by mouth rinsing. This drug delivery system also allows for the treatment of other diseases and disorders associated with the mouth.

Pharmaceutical agents to be delivered include therapeutic, nutritional, diagnostic, cosmeceutical, and prophylactic agents. The active agents can be small molecule active agents or biomacromolecules, such as proteins, polypeptides, carbohydrates or sugars, lipids, nucleic acids and oligonucleotide drugs (including DNA, RNAs, antisense, aptamers, small interfering RNAs, ribozymes, external guide sequences for ribonuclease P, and triplex forming agents), and combination molecules thereof. Suitable small molecule active agents include organic and organometallic compounds. The small molecule active agents can be a hydrophilic, hydrophobic, or amphiphilic compound.

Exemplary therapeutic agents that can be incorporated into particles include antibiotics, antiviral, anti-inflammatories, vaccines (tumor antigens), chemotherapeutics, growth factors and hormones, local anesthetics, and nutraceuticals such as vitamins and plant abstracts.

Exemplary cosmetic materials include agents that combat halitosis directly or indirectly, bleaching agents, coloring agents, and dyes

Exemplary diagnostic agents include paramagnetic molecules, fluorescent compounds, magnetic molecules, and radionuclides, x-ray imaging agents, and contrast agents and dyes.

The agents may also include flavorings, breath fresheners, and other excipients.

For whitening of teeth, a hydrogen peroxide producing material such as calcium peroxide can be encapsulated in the particle. Once calcium peroxide is released, it will react with water and form hydrogen peroxide. This system is advantageous since the bleaching agent, hydrogen peroxide, is not produced until calcium peroxide is released from the particle. Furthermore, calcium peroxide has a low water solubility, allowing it to be encapsulated in hydrophobic particles. To encapsulate other peroxides such as peroxymonosulfate, the tetra-n-butylammonium salt form can be encapsulated. Tetra-n-butylammonium peroxymonosulfate will be more hydrophobic than peroxymonosulfate and result in a higher encapsulation efficiency within hydrophobic particles. Similar strategies can be employed for peroxydisulfate and phthalimido-peroxy-hexanoic acid.

### III. Methods of Making Particles and Functionalizing

1. Methods of Making Particles

Particles can be prepared using many known methods. In some embodiments, the particles are prepared as described by Zhang, et al., ACS Nano. 2(8):1696-702 (2008). This method prepares a lipid-polymer hybrid particle with high drug encapsulation yield, tunable and sustained drug release profile, excellent serum stability, and potential for differential targeting of cells or tissues. The particles include three distinct functional components: (i) a hydrophobic polymeric core where poorly water-soluble drugs can be encapsulated; (ii) a hydrophilic polymeric shell with antibiofouling properties to enhance particle stability and systemic circulation half-life; and (iii) a lipid monolayer at the interface of the core and the shell that acts as a molecular fence to promote drug retention inside the polymeric core, thereby enhancing drug encapsulation efficiency, increasing drug loading yield, and controlling drug release. The particle is prepared by self-assembly through a single-step nanoprecipitation method in a reproducible and predictable manner.

In some embodiments, a hybrid particle system is engineered to have a hydrophobic drug-eluting core, a hydrophilic polymeric shell, and a lipid monolayer, as described by Chan, et al. Biomaterials 30, 1627-1634 (2009). Poly(ethylene glycol) (PEG) covalently conjugated to 1,2-distearoyl-sn-glycero-3-phosphoethanolamine (DSPE) is used to form the hydrophilic polymeric shell. To complete the lipid monolayer, soybean lecithin, which is considered Generally Regarded as Safe (GRAS), is used to form the core-shell interface.

A nanoparticulate system designed with an approximately 60 nm core-shell hybrid particle system consisting of a polymeric core, a lipid interface and a PEG corona formed of poly(lactic acid) (PLA) conjugates of paclitaxel made by a modified drug-alkoxide ring-opening strategy (Chamberlain, et al. J Am Chem Soc 123, 3229-3238 (2001); Dechy-Cabaret, et al. Chem Rev 104, 6147-6176 (2004)), allows for controlled drug release by gradual ester hydrolysis despite the large surface area and short diffusion distances in sub-100 nm particles. For the hydrophobic drug-eluting core, drug-polylactide conjugates can be synthesized by a drug/alkoxide-initiated ring-opening polymerization strategy. The particles can be functionalized with ligands (Peer, et al. Nat Nanotechnol 2, 751-760 (2007); Langer, R. Nature 392, 5-10 (1998)) to increase targeting specificity across a range of diseases in a consistent and reproducible manner. Drug elution rates can be further controlled by varying lactide/drug ratios during ring-opening polymerization, resulting in different PLA chain lengths attached to the drug.

Particles can be fabricated from different polymers using different methods as disclosed below.

Solvent Evaporation.

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting particles are washed with water and dried overnight in a lyophilizer. Particles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

Hot Melt Microencapsulation.

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between 0.5 to 1000 microns are obtained with this method. The external surfaces of spheres prepared with this technique are usually smooth and dense. This procedure is used to prepare particles made of polyesters and polyanhydrides. However, this method is limited to polymers with molecular weights between 1,000-50,000 Da.

Solvent Removal.

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

Spray-Drying.

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm. Particles ranging between 1-10 microns are obtained with a morphology which depends on the type of polymer used.

Hydrogel Particles.

Particles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The particles are left to incubate in the bath for twenty to thirty minutes in order to allow sufficient time for gelation to occur. Particle size is controlled by using various size extruders or varying either the nitrogen gas or polymer solution flow rates. Chitosan particles can be prepared by dissolving the polymer in acidic solution and crosslinking it with tripolyphosphate. Carboxymethyl cellulose (CMC) particles can be prepared by dissolving the polymer in acid solution and precipitating the particle with lead ions. In the case of negatively charged polymers (e.g., alginate, CMC), positively charged ligands (e.g., polylysine, polyethyleneimine) of different molecular weights can be ionically attached.

2. Functionalization of Particles

Improved functionality results in prolonged periods of release of encapsulated agent over the course of controlled release from the particle (days to weeks) at a site of attachment of the particles via the targeting molecule. Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in at least three ways. The first is during the preparation of the particles, for example during the emulsion preparation of particles by incorporation of stabilizers with functional chemical groups. A second is by conjugation of the targeting ligand or agent to a terminus of the polymer forming the particle, which is then formed so that targeting ligand is on the outside and agent to be delivered is on the outside, inside or both of the particle. In a third method, post-particle preparation, the polymeric particle is directly crosslinked using ligands with homo- or heterobifunctional crosslinkers. This may use a suitable chemistry and a class of crosslinkers (CDI, EDAC, glutaraldehydes, etc. as discussed in more detail below) or any other crosslinker that couples ligands to the particle surface via chemical modification of the particle surface after preparation Amphiphilic molecules such as fatty acids, lipids or functional stabilizers may be passively adsorbed and adhered to the particle surface, thereby introducing functional end groups for tethering to ligands.

Methods for coupling ligands to metals, ceramics and polymers are well known. In some embodiments, peptides are coupled to particles as described by Gu, et al., in Methods Mol. Biol. 544:589-5999 (2009), which describes the preparation of drug-encapsulated particles formulated with biocompatible and biodegradable poly(D:, L:-lactic-co-glycolic acid)-block-poly(ethylene glycol) (PLGA-b-PEG) copolymer and surface functionalized with the A10 2-fluoropyrimidine ribonucleic acid aptamers.

Other methods are well known. Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the particles, for example during the emulsion preparation of particles by incorporation of stabilizers with functional chemical groups. A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands. For example, approaches to introduce functionality into PLGA surfaces include synthesis of PLGA copolymers with amine (Lavik et al J Biomed Mater Res; 58(3):291-4 (2001); Caponetti et al. J Pharm Sci; 88(1):136-41 (1999)) or acid (Caponetti et al J Pharm Sci; 88(1):136-41 (1999)) end groups followed by fabrication into particles. Another approach involves the blending or adsorption of functional polymers such as polylysine (Faraasen et al. Pharm Res; 20(2):237-46 (2003); Zheng et al. Biotechnology Progress; 15(4):763-767 (1999)) or poly(ethylene-alt-maleic acid) (PEMA) (Keegan et al. Macromolecules (2004)) or PEG (Muller J Biomed Mater Res; 66A(1):55-61 (2003)) into PLGA and forming particles and matrices from these blends (Zheng, et al. 1999; Keegan, 2004; Park et al. J Biomater Sci Polym Ed; 9(2):89-110 (1998); Croll Biomacromolecules; 5(2):463-73 (2004); Cao et al. Methods Mol Biol; 238:87-112 (2004)). Plasma treatment of the PLGA matrix has also been proposed for the purpose of modifying its surface properties and introducing hydrophilic functional groups into the polymer (Yang et al. J Biomed Mater Res; 67A(4):1139-47 (2003); Wan et al., Biomaterials; 25(19):4777-83 (2004)). The most widely used coupling group is poly(ethylene glycol) (PEG), because this group creates a hydrophilic surface that facilitates long circulation of the particles.

Incorporating ligands in liposomes is easily achieved by conjugation to the phospholipid head group, in most cases phosphotidylethanolamine (PE), and the strategy relies either on a preinsertion of the functionalized lipid or post insertion into a formed liposome. Functionality can also be introduced by incorporating PEG with functional endgroups for coupling to target ligands. In a preferred embodiment, the targeting ligand is PEG.

One useful protocol involves the “activation” of hydroxyl groups on polymer chains with the agent, carbonyldiimidazole (CDI) in aprotic solvents such as DMSO, acetone, or THF. CDI foul's an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

A useful coupling procedure for attaching ligands with free thiol groups to polymers involves the use of polymers with maleimide end-groups. This method is useful for attaching peptides, nucleic acids and antibodies which are modified to contain cysteines (thiol groups) for conjugation to maleimide. Briefly, the activation involves reduction of disulfide bonds formed between cysteine thiol groups of ligands by a reducing agent, TCEP ((tris(2-carboxyethyl)phosphine)) in a oxygen-free environment, then adding the polymer (with maleimide end-group) to the reduced ligand. Activation and coupling take place at 1-10 mM EDTA at pH 6.5-7.5. The linkage is a covalent and stable linkage in the pH range of 1-8 once conjugation has taken place.

Any suitable coupling method known to those skilled in the art for the coupling of ligands and polymers with double bonds, including the use of UV crosslinking, may be used for attachment of molecules to the polymer. Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

### IV. Methods of Use

The particles can be applied as a dry powder, suspension, spray, lotion, gel, aerosol, paste, gum or by physical placement (painting or toothbrushing) The effective dosage will be determined by the amount of agent to be released, and the site where release is to occur. Suitable excipients are well known and commercially available from any compounding facility.

Representative applications include drug delivery to the oral cavity or mouth, throat or other place adjacent to exposed bone (as in periodontal disease) or teeth enamel Binding may be to the mucosa and/or to the teeth or bone. Multiple ligands, to mucosa as well as teeth or bone, may enhance delivery of agents where uptake through the mucosa is desired, or so that binding occurs in regions where the teeth or bone is in proximity with the mucosa (as in the case of periodontal disease. In addition to tooth whitening, this system can be applied to the treatment of various diseases by incorporation of different active agents.

For treatment of the teeth, it may be desirable to only have ligands binding to teeth. For example, hydroxyapatite-binding drug-loaded particles having targeting ligands immobilized to their surfaces through covalent bonds will bind to the tooth's surface, releasing agent at the site of binding. In a preferred embodiment, particles bind via hydroxyapatite-targeting bisphosphonate molecules, and continuously release whitener. This binding is reversible by administration of a solution that dissociates the targeting ligand from its receptor. In a preferred embodiment, pH is used to release the particles. Once unbound from the tooth surface, the particle can be rapidly eliminated by mouth rinsing.

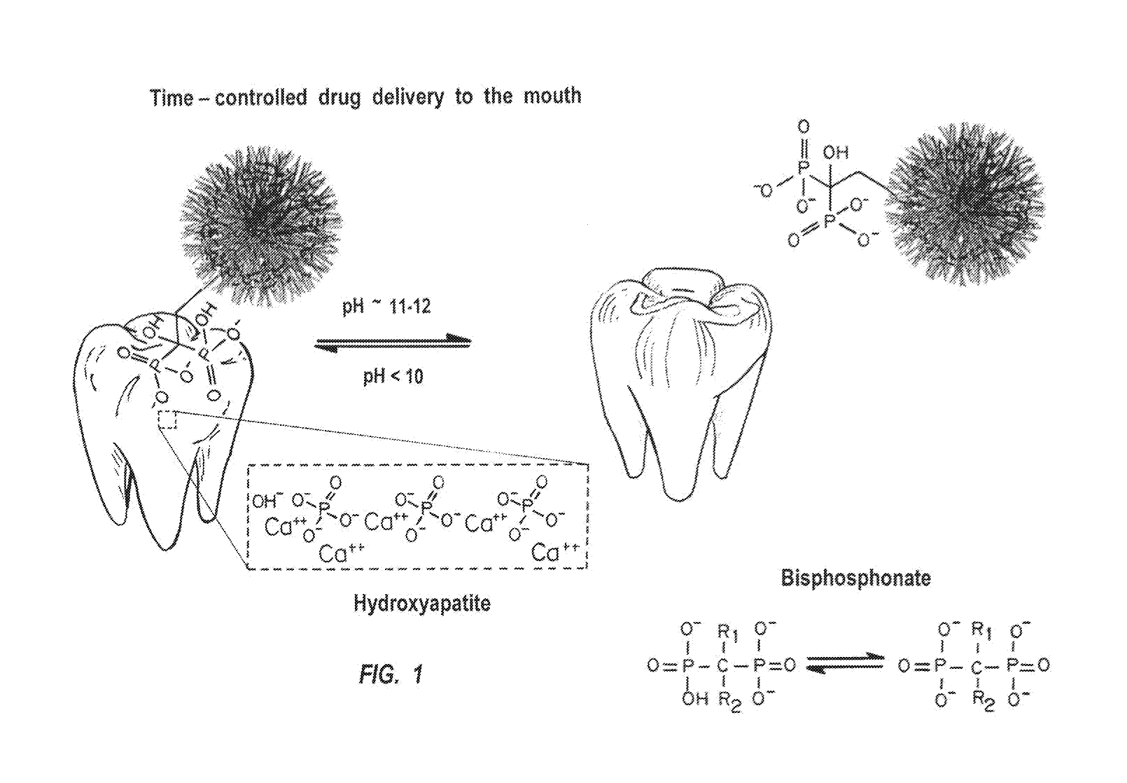

FIG. 1 is a schematic of the use of the particles to bleach teeth. The particles have a hydrophobic core and hydrophilic or amphiphilic corona, having selective binding ligands bound thereto. These bind to molecules such as the hydroxyapatite of teeth, and can be released by exposure to a high pH solution such as a pH 11-12.

The preferred pH range is between 9 and 10. The pH should be just above the range the mouth may normally experience. Saliva is 6.5 to 7.5 but some foods may have a pH of 9, and over the counter drugs such as milk of magnesia has a pH of about 10. Additional or alternative cleavable linkers may also be used, such as photolabile linkers to cause cleavage by light or bonds that are cleaved by specific enzymes

As demonstrated in the examples, alendronate coated particles rapidly bind to particle suspensions of hydroxyapatite. Particles can be mixed with different solutions before application to enhance particle binding (e.g. surfactants to help penetrate the mucus layers if that is a problem). Particles can also be introduced with a toothpaste-like gel to increase contact time between the particles and the tooth.

The present invention will be further understood by reference to the following non-limiting examples.

### EXAMPLES

### Example 1. Synthesis of Hydroxyapatite Targeted-Adhesive Particles; Binding and Release Kinetics

Materials and Methods

Materials: PLA-PEG-NHS, alendronate trihydrate (sigma), PLA-PEG-LRB (fluorescent dye), phosphate buffer, phosphate buffer saline, tris buffer, borate buffer, hydroxyapatite.

Synthesis of PLA-PEG-Alendronate Nanoparticles

24 mg of PLA-PEG-NHS (MW 5 kDa) was dissolved in 10 ml of dry acetonitrile. The solvent was evaporated in a round bottom flask producing a thin film. A solution of 15 mg of alendronate in phosphate buffer at pH 8.0 was added to the film at a temperature of 45° C. Mixture was left at room temperature for 4 hours, followed by dialysis (MWCO 3,500). A white powder was recovered after lyophilization.

Various particles containing a mixture of 80% PLA-PEG-Alendronate and 20% PLE-PEG-LRB for visualization were prepared by dissolving the polymers in acetonitrile following by evaporation to form a film. PBS at pH 7.4 was then added at 45° C. to form the particles.

Reversible Binding Experiments

1 ml of a suspension of 10 mg/ml of hydroxyapatite and 50 μg/ml of NPs (80% alendronate/20% LRB) was mixed for 12 hours (to allow binding). The hydroxyapatite was washed with 10 mM Tris buffer pH 7.5 (3×1 ml) to remove unbound particles. The particle solution was centrifuged and split into 2 equal samples (5 mg each). One sample was used as an internal control, while one was washed with high pH (10.5, 11 or 12). The sample was mixed with 500 ul of high pH buffer for 10 seconds, spun down to separate the solid HA from the supernatant. The solid HA was then washed with 10 mM Tris pH 7.5 to remove any unbound excess NPs The internal control was treated the same way, but was never exposed to high pH. The control was exposed to pH 7.5 and then washed with Tris pH 7.5. Minimal fluorescence was observed in the supernatant in the control, indicating no dissociation. Fluorescence of the HA pellets of the control and sample was used to calculate the dissociation degree (% removed from HA=fluorescence of HA sample pellet/fluorescence of HA control pellet*100). (n=4 for each condition.

Results

Polymeric nanoparticles with a hydrophobic core (PLA) and a PEG corona were synthesized for drug encapsulation. Bisphosphonates such as alendronate, neridronate and pamidronate are known to bind to hydroxyapatite. The particles were surface functionalized with alendronate targeting ligands, the resulting PLA-PEG-alendronate particles were labeled with LRB fluorescent dye, and their binding to hydroxyapatite powder was analyzed over different time periods. 50 μl of a 2.5 mg/ml particle solution was added to a 500 μl suspension of 25 mg/ml of hydroxyapatite. At set time points, the mixture was centrifuged and the hydroxyapatite pellet was washed with buffer to remove unbound particles.

Table 1 shows the kinetics of cleavage of PLA-PEG-Alendronate micelles from hydroxyapatite. At a pH of 12, >99% of particles bound to hydroxyapatite were released from the interaction. At a lower pH of 11, >90% micelles were released.

TABLE 1
Release from Hydroxyapatite by pH Change

| 10 seconds | pH 12 |
|---|---|
| % Fluorescence remaining on HA | 0.73 ± 0.12 |
| % Fluorescence removed from HA | 99.3 ± 0.12 |
| (100-pellet) | |
| 10 minutes | pH 11 |
| % Fluorescence remaining on HA | 8.8 ± 1.3 |
| % Fluorescence removed from HA | 91.2 ± 1.3 |
| (100-pellet) | |

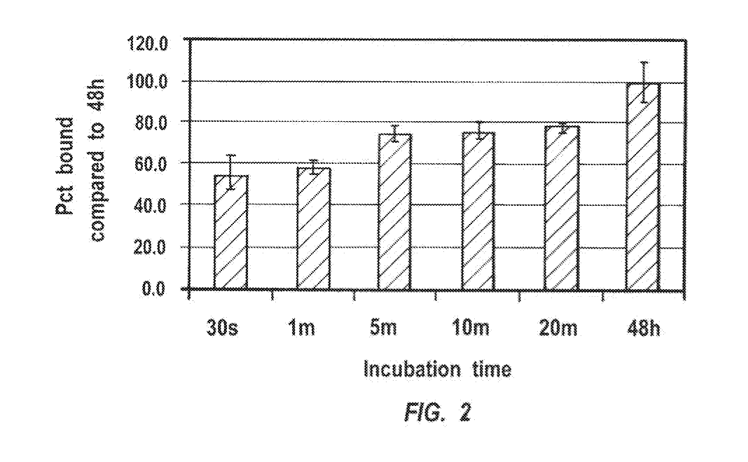

The group in which the micelles and hydroxyapatite were incubated together for 48 hours was defined to exhibit maximum binding. Micelle binding reached ˜50% of maximum binding within 30 seconds of incubation (FIG. 2). This indicated that considerable binding to the teeth could occur within a practical timeframe. Micelles without alendronate showed negligible binding even after 24 hours.

The results demonstrate that bisphosphonate binding to hydroxyapatite is reversible and pH dependent. At higher pH (˜11-12), alendronate functionalized particles detach from hydroxyapatite. Only ˜10 seconds are required for >99% of the particles to dissociate from hydroxyapatite at pH 12. At pH 11, the particles dissociate within 10 minutes. Dissociation (measured in pKa) is a result of complete deprotonation of the phosphate groups on alendronate. The highest pKa of the alendronate phosphate groups has been reported to be ˜10.96⁶, which is in good agreement with the disclosed particles unbinding experiments. Pamidronate's highest pKa is 10.18⁶, almost one unit lower than that of alendronate. This allows for detachment of particles at a lower pH (˜10) than that seen with alendronate functionalized particles.

Modifications and variations of the methods and compositions described herein will be obvious to those skilled in the art and are intended to come within the scope of the appended claims.

1. A composition comprising particles comprising
a core comprising a therapeutic, diagnostic, nutraceutical, cosmeceutical or prophylactic agent to be released;
a corona; and
targeting ligands immobilized to the corona via covalent bonding, wherein the targeting ligands selectively bind mucosa, bone or teeth.

2. The composition of claim 1 wherein the particles are polymeric.

3. The composition of claim 1, wherein the core is hydrophobic, and wherein the corona is hydrophilic.

4. The composition of claim 3, wherein the hydrophobic core comprises a polyhydroxy acid, polyanhydride, polyorthoester, polyhydroxyalkanoate, or a combination thereof.

5. The composition of claim 1, wherein the corona comprises a polyalkylene polymer or block copolymer.

6. The composition of claim 1, wherein the particles are liposomes or micelles.

7. The composition of claim 1, wherein the targeting ligands comprise bisphosphonate molecules.

8. The composition of claim 1, wherein the bisphosphonate is selected from the group consisting of alendronate, neridronate, and pamidronate.

9. The composition of claim 1, wherein the bisphosphonate molecules target hydroxyapatite to bind the particles to the teeth.

10. The composition of claim 1, wherein the targeting ligands selectively bind to mucosa.

11. The composition of claim 1, wherein the agent is released continuously.

12. The composition of claim 9, wherein the agent is a bleaching agent.

13. The composition of claim 12, wherein the bleaching agent is selected from the group consisting of peroxymonosulfate, peroxydisulfate, phthalimido-peroxy-hexanoic acid, and hydrogen peroxide.

14. The composition of claim 12, wherein the bleaching is produced by reaction of encapsulated calcium peroxide with an aqueous solution.

15. The composition of claim 13, wherein the particles are releasable by administration of a solution having a particular pH.

16. The composition of claim 14, wherein the solution has a pH of 10 or greater.

17. The composition of claim 1, wherein the ligands selectively bind mucosa.

18. (canceled)

19. The composition of claim 1, comprising a therapeutic agent.

20. The composition of claim 1, formulated for application as a dry powder, gel, spray, toothpowder or paste, gum, suspension, spray, lotion, aerosol, or by physical placement (e.g., painting or toothbrushing).

21. A method of selective delivery to the mouth or teeth comprising administering to the mouth or teeth the composition of claim 1.

22. The method of claim 21, wherein the tooth is contacted with the composition for 1 minute or less.

23. The method of claim 21, wherein the particles are released from the binding site by administration of a solution having a particular pH.

24. The method of claim 21, wherein the composition is applied to the teeth, the ligands selectively binding to hydroxyapatite, and the particles containing a compound reacting with an aqueous solution to release peroxide.

25. The method of claim 21, wherein the composition selectively binds to mucosa, teeth, or both, and contains therapeutic agent for treatment of periodontal disease.
